(19) United States
(12) Patent Application Publication
(10) Pub. No.: US 2005/0192298 A1
(43) Pub. Date: Sep. 1, 2005

(54) Crystal forms of E-2-Methoxy-N-(3-{4-[3-methyl-4-(6-methyl-pyridin-3-yloxy)-phenylamino]-quinazolin-6-yl}-allyl)-acetamide

(75) Inventors: Zheng Jane Li, Quaker Hill, CT (US); Jason Albert Leonard, Concord, NH (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/051,145

(22) Filed: Feb. 4, 2005

Related U.S. Application Data
(60) Provisional application No. 60/548,743, filed on Feb. 27, 2004.

(51) Int. Cl. A61K 31/517; C07D 43/02

(52) U.S. Cl. 514/266.21; 544/284

(57) ABSTRACT

Crystal forms of E-2-Methoxy-N-(3-{4-[3-methyl-4-(6-methyl-pyridin-3-yloxy)-phenylamino]-quinazolin-6-yl}-allyl)-acetamide. A crystal form is useful in the synthesis of salts and complexes of E-2-Methoxy-N-(3-{4-[3-methyl-4-(6-methyl-pyridin-3-yloxy)-phenylamino]-quinazolin-6-yl}-allyl)-acetamide. A crystal form also improves stability of tabletted or capsuled E-2-Methoxy-N-(3-{4-[3-methyl-4-(6-methyl-pyridin-3-yloxy)-phenylamino]-quinazolin-6-yl}-allyl)-acetamide as a drug product.

### BACKGROUND OF THE INVENTION

This invention relates to crystal forms of E-2-Methoxy-N-(3-{4-[3-methyl-4-(6-methyl-pyridin-3-yloxy)-phenylamino]-quinazolin-6-yl}-allyl)-acetamide having the formula I:

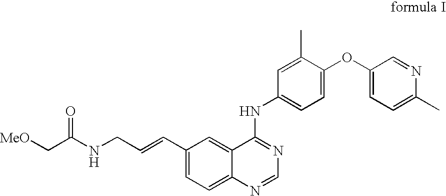

Formula I in its free base form is described in International Publication No. WO01/98277 published Dec. 27, 2001, the disclosure of which is hereby incorporated herein by reference in its entirety. The foregoing application is assigned in common with the present application. The free base of formula I is useful in the treatment of hyperproliferative diseases, such as cancers.

Succinate and malonate forms, including the sesquisuccinate and di-malonate forms of E-2-Methoxy-N-(3-{4-[3-methyl-4-(6-methyl-pyridin-3-yloxy)-phenylamino]-quinazolin-6-yl}-allyl)-acetamide were disclosed in U.S. Provisional Patent Application Ser. No. 60/340,885, filed Dec. 12, 2001, incorporated herein by reference.

Complexes and salts of E-2-Methoxy-N-(3-{4-[3-methyl-4-(6-methyl-pyridin-3-yloxy)-phenylamino]-quinazolin-6-yl}-allyl)-acetamide which may be prepared from a free base crystal form can be the active agent of pharmaceutical compositions as disclosed in U.S. patent application Ser. No. 10/738,972, filed Dec. 17, 2003, incorporated herein by reference. These complexes and salts are useful in the treatment of hyperproliferative diseases, such as cancers, in mammals, especially humans.

The aforementioned forms of formula I represent a significant advance in the art insofar as these forms of E-2-Methoxy-N-(3-{4-[3-methyl-4-(6-methyl-pyridin-3-yloxy)-phenylamino]-quinazolin-6-yl}-allyl)-acetamide are useful in the treatment of hyperproliferate diseases. It is thus important that the compound be amenable to the synthesis of the important salts and complexes of that compound. In addition, the product, employed as a drug, need be stable when produced in dosage forms.

### SUMMARY OF THE INVENTION

Crystal forms of E-2-Methoxy-N-(3-{4-[3-methyl-4-(6-methyl-pyridin-3-yloxy)-phenylamino]-quinazolin-6-yl}-allyl)-acetamide have now been discovered which are useful in the synthesis of salts and complexes of the titled compound. The use of these stable crystal forms improves the purity of the titled compound. These stable forms also alleviate stability problems associated with tabletted or capsuled forms of the compound as a drug product.

In accordance with the present invention, crystal forms of E-2-Methoxy-N-(3-{4-[3-methyl-4-(6-methyl-pyridin-3-yloxy)-phenylamino]-quinazolin-6-yl}-allyl)-acetamide having the following formula I: 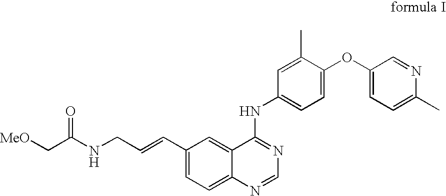
are disclosed.

The crystal form of the compound of the present invention includes Form A and hydrates and/or solvates thereof; Form B and hydrates and/or solvates thereof; Form C and hydrates and/or solvates thereof; Form F and hydrates and/or solvates thereof; Form G and hydrates and/or solvates thereof, and Form H and hydrates and/or solvates thereof.

### BRIEF DESCRIPTION OF THE DRAWINGS

The present invention may be better understood by references to the drawings of which:

FIG. 1 is a diffractogram of Crystal Form A of E-2-Methoxy-N-(3-{4-[3-methyl-4-(6-methyl-pyridin-3-yloxy)-phenylamino]-quinazolin-6-yl}-allyl)-acetamide;

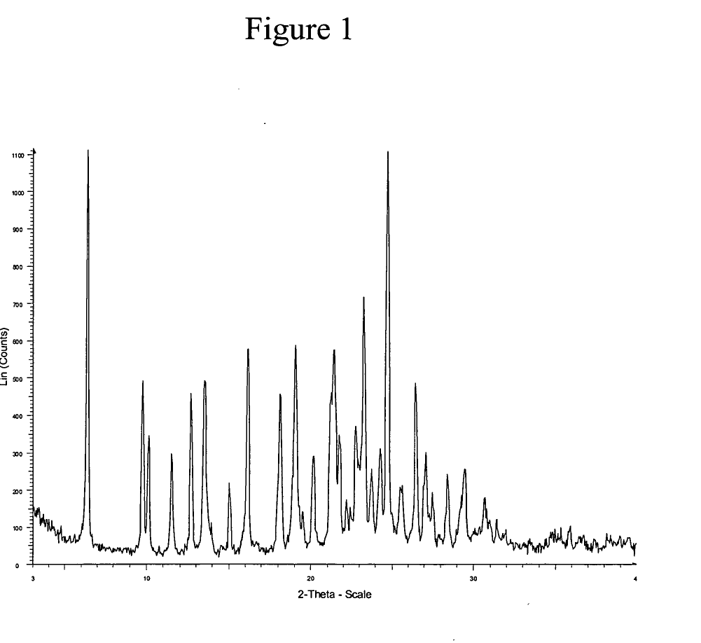

FIG. 2 is a diffractogram of Crystal Form B of E-2-Methoxy-N-(3-{4-[3-methyl-4-(6-methyl-pyridin-3-yloxy)-phenylamino]-quinazolin-6-yl}-allyl)-acetamide;

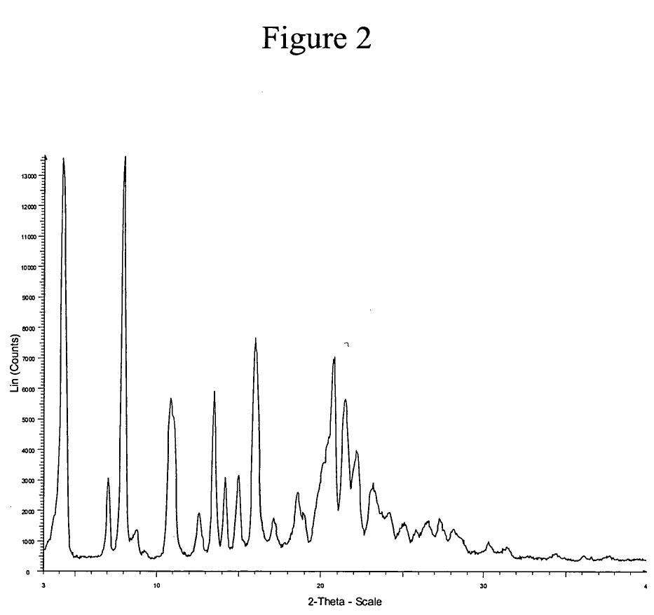

FIG. 3 is a diffractogram of Crystal Form C of E-2-Methoxy-N-(3-{4-[3-methyl-4-(6-methyl-pyridin-3-yloxy)-phenylamino]-quinazolin-6-yl}-allyl)-acetamide;

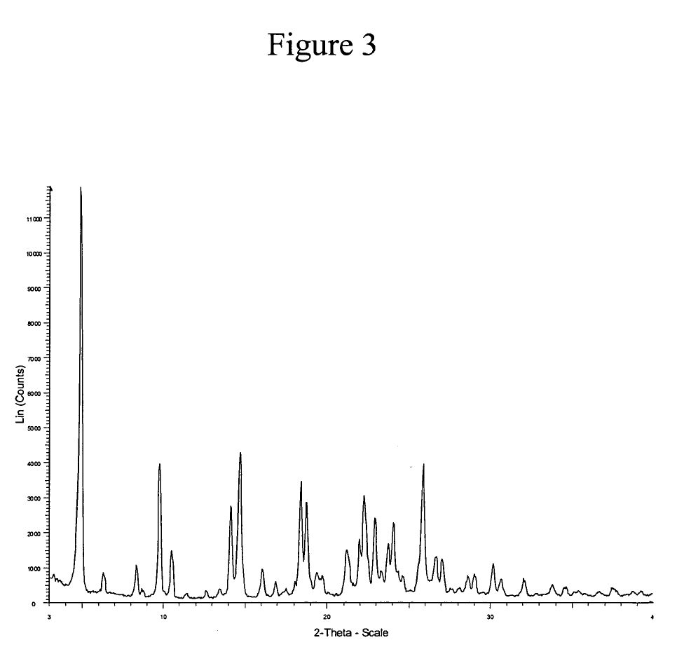

FIG. 4 is a diffractogram of Crystal Form F of E-2-Methoxy-N-(3-{4-[3-methyl-4-(6-methyl-pyridin-3-yloxy)-phenylamino]-quinazolin-6-yl}-allyl)-acetamide;

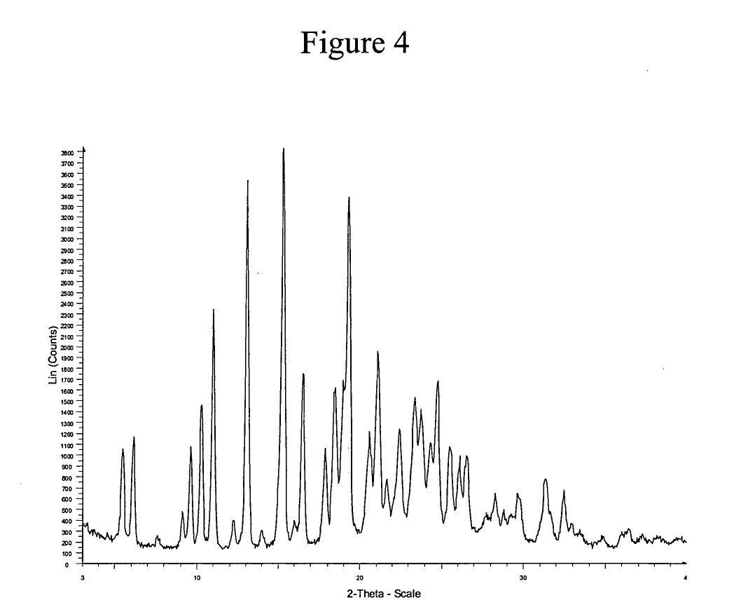

FIG. 5 is a diffractogram of Crystal Form G of E-2-Methoxy-N-(3-{4-[3-methyl-4-(6-methyl-pyridin-3-yloxy)-phenylamino]-quinazolin-6-yl}-allyl)-acetamide;

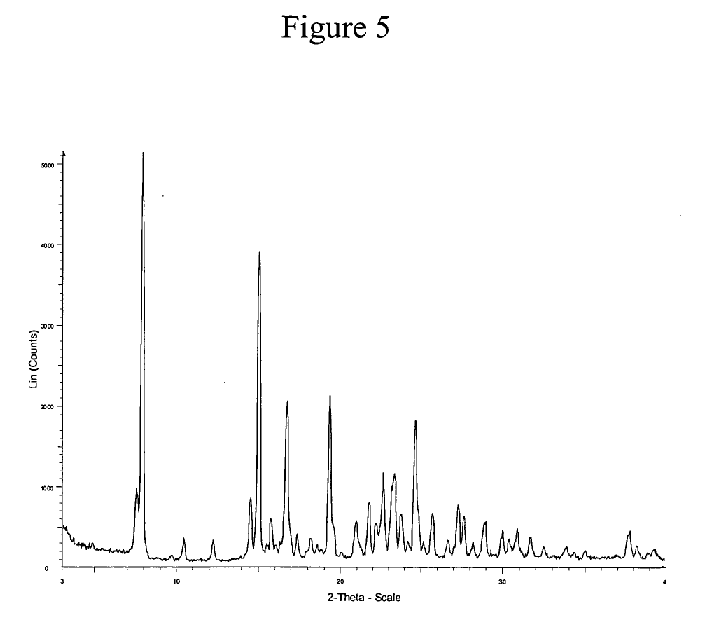

FIG. 6 is a diffractogram of Crystal Form H of E-2-Methoxy-N-(3-{4-[3-methyl-4-(6-methyl-pyridin-3-yloxy)-phenylamino]-quinazolin-6-yl}-allyl)-acetamide; and

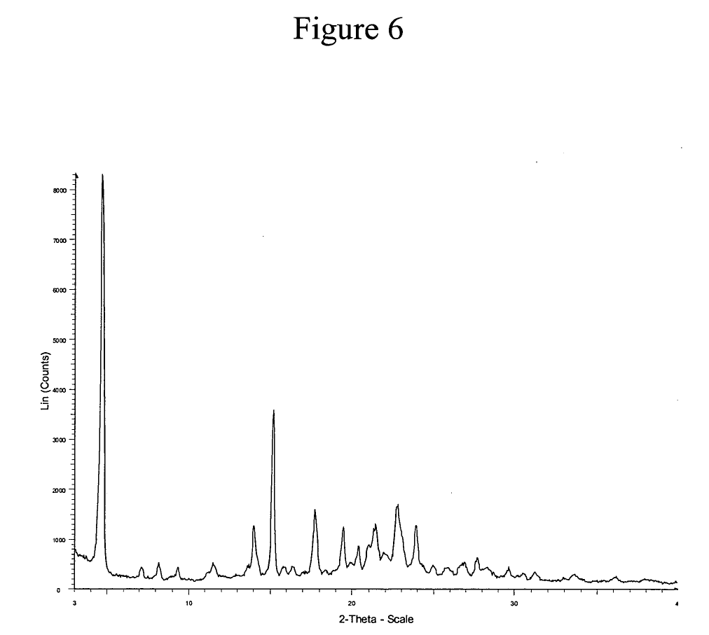

FIG. 7 is an solid state nmr spectra of Crystal Form A of E-2-Methoxy-N-(3-{4-[3-methyl-4-(6-methyl-pyridin-3-yloxy)-phenylamino]-quinazolin-6-yl}-allyl)-acetamide.

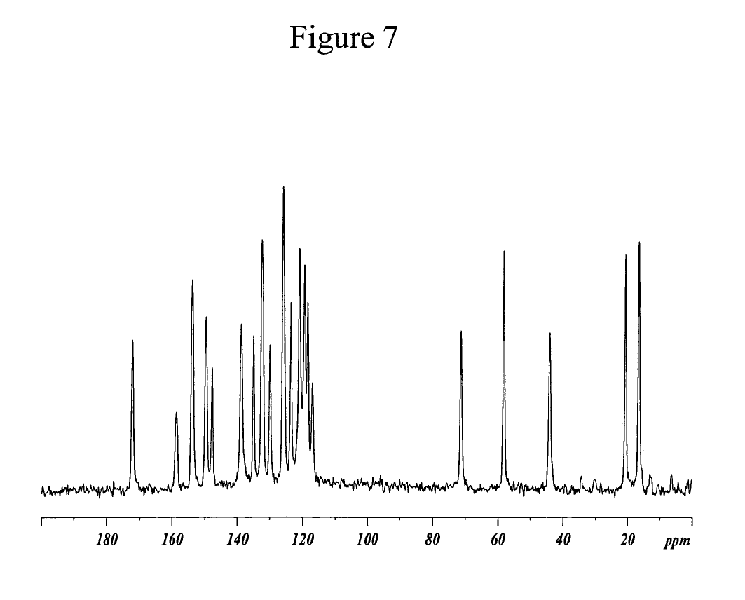

### DETAILED DESCRIPTION

The present invention relates to crystal forms of E-2-Methoxy-N-(3-{4-[3-methyl-4-(6-methyl-pyridin-3-yloxy)-phenylamino]-quinazolin-6-yl}-allyl)-acetamide having the formula I:

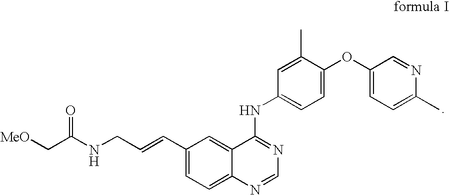

Although the present invention is directed to crystal forms of the E-2-Methoxy-N-(3-{4-[3-methyl-4-(6-methyl-pyridin-3-yloxy)-phenylamino]-quinazolin-6-yl}-allyl)-acetamide, in a preferred embodiment, six crystal forms are provided. Those skilled in the art are aware that other forms may be synthesized by modifications to the synthesis methods employed in preparing the crystal forms disclosed herein. These forms are characterized by their powder x-ray diffraction patterns. These patterns have been identified on a Bruker D5000 diffractometer using copper radiation (wavelength 1:1.54056). The tube voltage and amperage were set to 40 kV and 50 mA, respectively. The divergence and scattering slits were set at 1 mm, and the receiving slit was set at 0.6 mm. Diffracted radiation was detected by a Kevex PSI detector. A theta-two theta continuous scan at 2.4°/min (1 sec/0.04° step) from 3.0 to 40°2θ was used. An alumina standard was analyzed to check the instrument alignment. Data were collected and analyzed using Bruker axis software Version 7.0. Samples were prepared by placing them in a quartz holder. It should be noted that Bruker Instruments purchased Siemans; thus, Bruker D5000 instrument is essentially the same as a Siemans D5000.

General synthetic methods which may be referred to for preparing substituted bicyclic derivations of which the crystal forms of the present invention relate are provided in WO 01/98277 (published Dec. 27, 2001), U.S. Pat. No. 5,747,498 (issued May 5, 1998), U.S. patent application Ser. No. 08/953,078 (filed Oct. 17, 1997), WO 98/02434 (published Jan. 22, 1998), WO 98/02438 (published Jan. 22, 1998), WO 96/40142 (published Dec. 19, 1996), WO 96/09294 (published Mar. 6, 1996), WO 97/03069 (published Jan. 30, 1997), WO 95/19774 (published Jul. 27, 1995) and WO 97/13771 (published Apr. 17, 1997). Additional procedures are referred to in U.S. patent application Ser. No. 09/488,350 (filed Jan. 20, 2000) and Ser. No. 09/488,378 (filed Jan. 20, 2000). The foregoing patents and patent applications are incorporated herein by reference in their entirety. Certain starting materials may be prepared according to methods familiar to those skilled in the art and certain synthetic modifications may be done according to methods familiar to those skilled in the art. A standard procedure for preparing 6-iodoquinazolinone is provided in Stevenson, T. M., Kazmierczak, F., Leonard, N. J., J. Org. Chem. 1986, 51, 5, p. 616. Palladium-catalyzed boronic acid couplings are described in Miyaura, N., Yanagi, T., Suzuki, A. Syn. Comm. 1981, 11, 7, p. 513. Palladium catalyzed Heck couplings are described in Heck et. al. Organic Reactions, 1982, 27, 345 or Cabri et. al. in Acc. Chem. Res. 1995, 28, 2. For examples of the palladium catalyzed coupling of terminal alkynes to aryl halides see: Castro et. al. J. Org. Chem. 1963, 28, 3136. or Sonogashira et. al. Synthesis, 1977, 777. Terminal alkyne synthesis may be performed using appropriately substituted/protected aldehydes as described in: Colvin, E. W. J. et. al. Chem. Soc. Perkin Trans. I, 1977, 869; Gilbert, J. C. et. al. J. Org. Chem., 47, 10, 1982; Hauske, J. R. et. al. Tet. Lett., 33, 26, 1992, 3715; Ohira, S. et. al. J. Chem. Soc. Chem. Commun., 9, 1992, 721; Trost, B. M. J. Amer. Chem. Soc., 119, 4, 1997, 698; or Marshall, J. A. et. al. J. Org. Chem., 62, 13, 1997, 4313.

A first crystal form of E-2-Methoxy-N-(3-{4-[3-methyl-4-(6-methyl-pyridin-3-yloxy)-phenylamino]-quinazolin-6-yl}-allyl)-acetamide is Form A. Form A is characterized by its intensities and peak locations of diffraction lines as set forth in Tables 1 to 3. These tables lists 2-theta values for the anhydride of Form A. However, hydrates and solvates are also within the scope of the present invention.

The most pronounced intensities and peak locations of diffraction lines are set forth in Table 1. It is observed that the experimental error in positions as to all Tables herein is +/−0.2 Theta.

TABLE 1

| 2 Theta (degree) | Rel (%) Intensity |
|---|---|
| 6.3 | 100 |
| 16.1 | 51.7 |
| 19.0 | 52.6 |
| 23.2 | 64.4 |
| 24.7 | 99.6 |

More pronounced intensities and peak locations of diffraction lines of Form A are set forth in Table 2.

TABLE 2

| 2 Theta (degree) | Rel (%) Intensity |
|---|---|
| 6.3 | 100.0 |
| 9.7 | 44.2 |
| 13.5 | 44.2 |
| 16.1 | 51.7 |
| 19.0 | 52.6 |
| 21.2 | 41.3 |
| 21.4 | 51.6 |
| 23.2 | 64.4 |
| 24.7 | 99.6 |
| 26.5 | 43.7 |

The intensities and peak locations of pronounced diffraction lines of Form A are set forth in Table 3.

TABLE 3

| 2-Theta (degree) | Rel. % intensity |
|---|---|
| 6.3 | 100.0 |
| 9.7 | 44.2 |
| 10.1 | 30.9 |
| 11.5 | 26.6 |
| 12.6 | 41.1 |
| 13.5 | 44.2 |
| 15.0 | 19.5 |
| 16.1 | 51.7 |
| 18.1 | 41.0 |
| 19.0 | 52.6 |
| 19.5 | 12.6 |
| 20.1 | 25.9 |
| 21.2 | 41.3 |
| 21.4 | 51.6 |
| 21.7 | 31.0 |
| 22.2 | 15.4 |
| 22.8 | 33.4 |
| 23.2 | 64.4 |
| 23.7 | 22.9 |
| 24.3 | 27.9 |
| 24.7 | 99.6 |
| 25.6 | 18.0 |
| 26.5 | 43.7 |
| 27.1 | 26.9 |
| 27.5 | 17.2 |
| 28.4 | 21.7 |
| 29.5 | 23.0 |
| 30.7 | 16.1 |

In addition Form A is characterized by single crystal X-ray analysis. Single crystal X-ray analysis data is obtained at room temperature using a Bruker X-ray diffractometer equipped with copper radiation and graphite monochromators. Structures were solved using direct methods. The SHELXTL computer library provided by Bruker AXS, Inc facilitated all necessary crystallographic computations and molecular displays.

The single crystal X-ray analysis of Form A is as follows:

Form A

| Empirical formula | C₂₇H₂₇N₅O₃ |
|---|---|
| Formula weight | 469.54 |
| Crystal size (mm) | 0.24 × 0.08 × 0.06 |
| Space group | P2₁(1)/n monoclinic |
| Unit cell dimensions | a = 9.456 Å |
| | b = 9.237 Å |
| | c = 27.947 Å |
| | α = 90° |
| | β = 92.97° |
| | γ = 90° |
| Z (per formula) | 4 |
| Density (g/cm³) | 1.279 |
| R | 0.0664 |

To compare the results between a single crystal and a powder sample, a calculated powder pattern can be obtained from single crystal results. The XFOG and XPOW computer programs provided as part of the SHELXTL computer library can perform this calculation. The SHELXTL computer library has been developed and upgraded over a long period of time.

The most recent version of this work in progress is as follows: SHELXTL™ Reference Manual, Version 5.1, Bruker AXS, Madison, Wis., USA (1997). Comparing the calculated powder pattern with the experimental powder pattern confirms whether a powder sample corresponds to an assigned single crystal structure. The results are displayed in the overlaid powder X-ray diffraction patterns. The lower pattern corresponds to the calculated powder pattern (from single crystal results) and the upper pattern corresponds to a representative experimental powder pattern. A match between the two patterns indicated the agreement between powder sample and the corresponding single crystal structure.

The solid state nuclear magnetic resonance (ss nmr) data for Form A is characterized by a spectra having the characteristics set forth in Table 4.

TABLE 4

| Peak # | ppm |
|---|---|
| 1 | 172.1 |
| 2 | 158.6 |
| 3 | 153.7 |
| 4 | 149.6 |
| 5 | 147.7 |
| 6 | 138.8 |
| 7 | 135.0 |
| 8 | 132.4 |
| 9 | 129.9 |
| 10 | 125.8 |
| 11 | 123.6 |
| 12 | 120.9 |
| 13 | 119.4 |
| 14 | 118.4 |
| 15 | 117.1 |
| 16 | 71.3 |
| 17 | 58.1 |
| 18 | 44.0 |
| 19 | 20.7 |
| 20 | 16.6 |

### Experimental Error +/−0.1 ppm

The above spectra was obtained by a procedure in which approximately 80 mg of sample were tightly packed into a 4 mm ZrO spinner for each sample analyzed. All spectra were collected at 295 K and ambient pressure on a Bruker-Biospin 4 mm BL CPMAS probe positioned into a wide-bore Bruker-Biospin Avance DSX 500 MHz NMR spectrometer. The samples were positioned at the magic angle and spun at 15.0 kHz, corresponding to the maximum specified spinning speed for the 4 mm spinners. The fast spinning speed minimized the intensities of the spinning side bands. ¹³C solid state spectra were collected using a proton decoupled cross-polarization magic angle spinning experiment (CPMAS). The proton decoupling field of approximately 85 kHz was applied. The number of scans (600) was adjusted to obtain adequate signal-to-noise (S/N). The recycle delay was adjusted to 6 seconds. The spectra were referenced using an external standard of crystalline adamantane, setting its upfield resonance to 29.5 ppm. The low intensity peaks in the spectrum at 34.3, 30.3, 28.3 as well as all peaks at chemical shifts smaller than 15 ppm are speinning side bands.

A second crystal form of E-2-Methoxy-N-(3-{4-[3-methyl-4-(6-methyl-pyridin-3-yloxy)-phenylamino]-quinazolin-6-yl}-allyl)-acetamide is Form B. The 2-theta values and intensities of Form B, as a hydrate or a solvate, is forth in Table 5. The present invention also contemplates the anhydrous form.

TABLE 5

| 2-Theta (degree) | Rel. % intensity |
|---|---|
| 4.1 | 99.5 |
| 7.0 | 22.4 |
| 7.9 | 100.0 |
| 8.7 | 9.8 |
| 10.8 | 41.5 |
| 11.0 | 37.2 |
| 12.5 | 14.1 |
| 13.5 | 43.2 |
| 14.1 | 22.5 |
| 15.0 | 23.1 |
| 16.0 | 56.0 |
| 17.1 | 12.7 |
| 18.6 | 18.8 |
| 19.0 | 14.2 |
| 20.1 | 26.2 |
| 20.8 | 51.5 |
| 21.5 | 41.4 |
| 22.2 | 29.1 |
| 23.2 | 21.2 |
| 24.2 | 14.1 |
| 25.1 | 11.9 |
| 25.8 | 10.1 |
| 26.6 | 12.1 |
| 27.3 | 12.8 |
| 28.1 | 10.0 |

A third crystal form of E-2-Methoxy-N-(3-{4-[3-methyl-4-(6-methyl-pyridin-3-yloxy)-phenylamino]-quinazolin-6-yl}-allyl)-acetamide is Form C. As in Forms A and B, Form C is identified by the intensities and peak locations of diffraction lines for that form, its hydrates and/or its solvates. The 2-theta values and intensities of a hydrate of Form C is set forth in Tables 6 to 8. However, anhydrous Form C and solvates thereof are also within the contemplation of the present invention.

Tables 6 to 8 present most pronounced, more pronounced and pronounced, respectively, 2-theta values and intensities of Form C.

TABLE 6

| 2-Theta (Degree) | Rel. % Intensity |
|---|---|
| 4.8 | 100.0 |
| 9.7 | 33.2 |
| 14.6 | 35.9 |
| 18.4 | 29.1 |
| 25.9 | 33.2 |

TABLE 7

| 2-Theta (Degree) | Rel. % Intensity |
|---|---|
| 4.8 | 100.0 |
| 9.7 | 33.2 |
| 14.1 | 23.1 |
| 14.6 | 35.9 |
| 18.4 | 29.1 |
| 18.7 | 24.0 |
| 22.3 | 25.7 |
| 22.9 | 20.1 |
| 24.0 | 19.0 |
| 25.9 | 33.2 |

TABLE 8

| 2-Theta (degree) | Rel. % intensity |
|---|---|
| 4.8 | 100.0 |
| 8.3 | 8.9 |
| 9.7 | 33.2 |
| 10.5 | 12.5 |
| 14.1 | 23.1 |
| 14.6 | 35.9 |
| 16.1 | 7.9 |
| 16.9 | 4.9 |
| 18.4 | 29.1 |
| 18.7 | 24.0 |
| 19.4 | 7.1 |
| 19.7 | 6.3 |
| 21.2 | 12.5 |
| 22.0 | 15.1 |
| 22.3 | 25.7 |
| 22.9 | 20.1 |
| 23.7 | 14.1 |
| 24.0 | 19.0 |
| 24.6 | 6.3 |
| 25.9 | 33.2 |
| 26.7 | 11.0 |
| 27.0 | 10.4 |
| 28.6 | 6.5 |
| 29.0 | 6.7 |
| 30.2 | 9.3 |
| 30.7 | 5.5 |
| 32.1 | 5.7 |
| 33.8 | 4.2 |

A fourth crystal form of the E-2-Methoxy-N-(3-{4-[3-methyl-4-(6-methyl-pyridin-3-yloxy)-phenylamino]-quinazolin-6-yl}-allyl)-acetamide is Form F. Form F is also identified by the intensities and peak locations of diffraction lines for a hydrate form in Tables 9 to 11. However, anyhydrous Form F and solvates are within the contemplation of the present invention.

Tables 9 to 11 present most pronounced, more pronounced and pronounced, respectively, 2-theta values and intensities of Form F.

TABLE 9

| 2-Theta (Degree) | Rel. % Intensity |
|---|---|
| 10.9 | 61.0 |
| 13.1 | 92.1 |
| 15.3 | 100.0 |
| 19.3 | 88.0 |
| 21.1 | 50.8 |

TABLE 10

| 2-Theta (Degree) | Rel. % Intensity |
|---|---|
| 10.2 | 38.0 |
| 10.9 | 61.0 |
| 13.1 | 92.1 |
| 15.3 | 100.0 |
| 16.5 | 45.5 |
| 18.5 | 42.2 |
| 19.0 | 44.0 |
| 19.3 | 88.1 |
| 21.1 | 50.8 |
| 23.3 | 39.8 |

TABLE 11

| 2-Theta (degree) | Rel. % intensity |
|---|---|
| 5.4 | 27.4 |
| 6.1 | 30.4 |
| 9.6 | 27.9 |
| 10.2 | 38.0 |
| 10.9 | 61.0 |
| 13.1 | 92.1 |
| 15.3 | 100.0 |
| 16.5 | 45.5 |
| 17.9 | 27.6 |
| 18.5 | 42.2 |
| 19.0 | 44.0 |
| 19.3 | 88.1 |
| 20.6 | 31.7 |
| 21.1 | 50.8 |
| 21.7 | 20.2 |
| 22.5 | 32.2 |
| 23.3 | 39.8 |
| 23.8 | 36.8 |
| 24.4 | 29.0 |
| 24.7 | 43.8 |
| 25.5 | 28.0 |
| 26.1 | 25.7 |
| 26.6 | 25.7 |
| 31.3 | 20.2 |
| 32.5 | 17.4 |

A fifth crystal form of the E-2-Methoxy-N-(3-{4-[3-methyl-4-(6-methyl-pyridin-3-yloxy)-phenylamino]-quinazolin-6-yl}-allyl)-acetamide is Form G. Form G, like the above discussed forms, is characterized in Tables 12 to 14. Specifically, a hydrate of Form G is provided therein. However, anhydrous Form G and solvates of Form G are within the scope of the present invention.

Tables 12 to 14 present most pronounced, more pronounced and pronounced, respectively, 2-theta values and intensities of Form G.

TABLE 12

| 2-Theta (Degree) | Rel. % Intensity |
|---|---|
| 7.8 | 100.0 |
| 15.0 | 75.9 |
| 16.7 | 40.0 |
| 19.3 | 41.2 |
| 24.7 | 35.2 |

TABLE 13

| 2-Theta (Degree) | Rel. % Intensity |
|---|---|
| 7.8 | 100.0 |
| 14.5 | 16.8 |
| 15.0 | 75.9 |
| 16.7 | 40.0 |
| 19.3 | 41.2 |
| 21.8 | 15.5 |
| 22.7 | 22.7 |
| 23.2 | 19.9 |
| 23.3 | 22.5 |
| 24.7 | 35.2 |

TABLE 14

| 2-Theta (degree) | Rel. % intensity |
|---|---|
| 7.8 | 100.0 |
| 10.4 | 6.9 |
| 12.2 | 6.4 |
| 14.5 | 16.8 |
| 15.0 | 75.9 |
| 15.8 | 11.7 |
| 16.7 | 40.0 |
| 17.4 | 7.8 |
| 18.2 | 6.8 |
| 19.3 | 41.2 |
| 21.0 | 11.1 |
| 21.8 | 15.5 |
| 22.2 | 10.5 |
| 22.7 | 22.7 |
| 23.2 | 19.9 |
| 23.3 | 22.5 |
| 23.8 | 12.7 |
| 24.7 | 35.2 |
| 25.7 | 13.0 |
| 26.7 | 6.4 |
| 27.3 | 14.9 |
| 27.6 | 12.1 |
| 28.2 | 6.0 |
| 28.9 | 10.8 |
| 30.0 | 8.8 |
| 30.9 | 9.2 |
| 31.7 | 7.2 |
| 37.8 | 8.7 |

A sixth crystal form of the E-2-Methoxy-N-(3-{4-[3-methyl-4-(6-methyl-pyridin-3-yloxy)-phenylamino]-quinazolin-6-yl}-allyl)-acetamide is Form H. Crystal Form H of the E-2-Methoxy-N-(3-{4-[3-methyl-4-(6-methyl-pyridin-3-yloxy)-phenylamino]-quinazolin-6-yl}-allyl)-acetamide free base is characterized by the intensities and peak locations of diffraction lines summarized in Tables 15 to 17. Specifically, anhydrous Form H, its hydrate or solvate is set forth in Tables 15 to 17. The anhydrous, hydrates and solvates of Form H are within the contemplation of the present invention.

Tables 15 to 17 present most pronounced, more pronounced and pronounced, respectively, 2-theta values and intensities of Form H.

TABLE 15

| 2-Theta (Degree) | Rel. % Intensity |
|---|---|
| 4.6 | 100.0 |
| 15.1 | 42.9 |
| 17.7 | 19.1 |
| 21.4 | 15.7 |
| 22.7 | 19.8 |

TABLE 16

| 2-Theta (Degree) | Rel. % Intensity |
|---|---|
| 4.6 | 100.0 |
| 14.0 | 15.1 |
| 15.1 | 42.9 |
| 17.7 | 19.1 |
| 19.4 | 15.0 |
| 20.4 | 10.0 |
| 21.0 | 10.5 |
| 21.4 | 15.7 |
| 22.7 | 19.8 |
| 23.9 | 15.4 |

TABLE 17

| 2-Theta (degree) | Rel. % intensity |
|---|---|
| 4.6 | 100.0 |
| 7.1 | 5.1 |
| 8.1 | 6.2 |
| 9.3 | 5.2 |
| 11.5 | 6.3 |
| 14.0 | 15.1 |
| 15.1 | 42.9 |
| 15.8 | 5.2 |
| 16.4 | 5.3 |
| 17.7 | 19.1 |
| 19.4 | 15.0 |
| 20.4 | 10.1 |
| 21.0 | 10.5 |
| 21.4 | 15.7 |
| 21.9 | 8.8 |
| 22.7 | 19.8 |
| 23.9 | 15.4 |
| 25.0 | 5.6 |
| 25.8 | 5.2 |
| 26.9 | 6.4 |
| 27.7 | 7.5 |
| 29.7 | 5.2 |
| 30.6 | 3.7 |
| 31.2 | 4.1 |
| 33.6 | 3.5 |

Solvate is defined as a crystal form of the titled compound that includes a solute ion or molecule with one or more solvent molecules.

Hydrate is defined as a compound formed by water with the titled compound.

Composition is defined as a crystal form of the titled compound with one or more other constituents.

Free base is defined as a basic form of an organic amine capable of forming acid salts such as hydrochlorides. Free base formed of Formula I are fully described in International Publication No. WO 01498277, incorporated herein by reference in its entirety.

Pharmaceutical composition is defined as a crystal form of the titled compound which includes a conventional pharmaceutical carrier or excipient. In addition, the composition may include other medicinal or pharmaceutical agents, carriers, adjuvants and the like. The pharmaceutical composition may be in a form suitable for oral administration as a tablet, a capsule, a pill, a powder, a sustained release formulation, a solution, a suspension, a sterile solution, suspension or emulsion for parenteral injection, an ointment or cream for topical administration or a suppository for rectal administration. The pharmaceutical composition may be in unit dosage forms suitable for single administration of precise dosages.

The following examples are provided to illustrate the present invention. Because these examples are given for illustrative purposes only the invention should not be deemed limited thereto.

### EXAMPLE 1

### Crystal Form A of E-2-Methoxy-N-(3-{4-[3-methyl-4-(6-methyl-pyridin-3-yloxy)-phenylamino]-quinazolin-6-yl}-allyl)-acetamide

### Synthesis of 6-Iodo-[3-methyl-4-(6-methyl-pyridine-3-yloxy)-phenylamino]-quinazoline

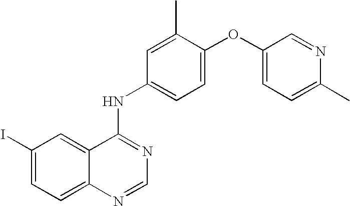

A 3 neck round bottom flask was fitted with a mechanical stirrer and kept under N₂. The flask was charged with the 6-iodo-4-chloroquinazoline (10.0 g, 34.43 mol) and dry THF (35 ml). Thereafter, 3-methyl-4-(6-methyl-pyridine-3-yloxy)-phenylamine (7.38 g, 34.43 mmol) and dry THF (45 ml) were added and the yellow suspension was heated to reflux. After 15 minutes most of the reactants went into solution and a fine yellow suspension was obtained. After 25 minutes, the internal temperature of the reaction mixture was 56° C., and precipitation of the desired product started. Heating was continued for a further 2 hours and the reaction mixture was allowed to cool to room temperature while remaining in the oil bath. Yellow crystals were collected by filtration, washed with cold (0° C.) THF (1×10 ml) and dried at 50° C., p<200 mbar. The title compound was obtained as light yellow crystals (15.75 g, 98%). Rf=0.45 (EtOAc/MeOH=9/1). ¹H NMR (CDCl₃, 300 MHz): δ=11.40 (br, s, 1H, NH), 9.29 (d, J=Hz, 1H, H-2), 8.91 (s, 1H, H-2″), 8.36-8.32 (m, 2H, H-7, H-8), 7.74-7.73 (m, 2H, H-4″, H-5), 7.62 (dd, J₁=8.7 Hz, J₂=2.6 Hz, 1H, H-5″) 7.49-7.46 (m, 2H, H-6′, H-5), 7.06 (d, J=8.7 Hz, 1H, H-2′), 2.54 (s, 3H, CH₃), 2.26 (s, 3H, CH₃). ¹³C NMR (CDCl₃+D₆-DMSO, 75 MHz): δ=159.51, 153.63, 153.17, 152.82, 152.70, 145.26, 141.37, 138.01, 134.75, 134.65, 131.05, 129.10, 128.74, 126.77, 124.86, 124.43, 120.41, 116.98, 94.89, 23.54, 17.67.

The title compound had a t_{R} (min) of 12.13 under the following RP-HPLC conditions: Symmetry Shield RP18, 75×4.6 mm; Flow 1.0 mL/min; 205/210/220/245 nm; Temp. 25° C.; Injection Volume: 10 μL of a ca. 0.5% solution in ACN/H₂O 9/1; Eluent: B: ACN, C, 0.01 mmol NH₄OAc in H₂O pH=6.0; and Gradient: 0 min: B=30%, C=70%; and 20 min: B=85%, C=15%.

### Synthesis of 2-Methoxy-acetic acid propargylamide

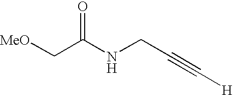

A solution of methoxy acetyl chloride (12.5 ml, 0.137 mol, 1.2 equiv.) in dry CH₂Cl₂ (45 ml) kept under N₂ was cooled to −40° C. A solution of propargylamine (7.98 ml, 0.125 mol, 1.0 equiv.) in dry CH₂Cl₂ (40 ml) was added over 45 minutes keeping the temperature less than −25° C. After 15 minutes triethylamine (17.4 ml, 0.125 mol, 1.0 equiv.) was added over 45 minutes keeping the temperature less than −25° C. The reaction mixture was warmed to room temperature. TLC after 3 hours showed conversion complete. The reaction mixture was quenched with H₂O (50 ml) and the organic phase was washed with half-saturated NaCl solution, filtered through cotton wool and concentrated at a temperature of 40° C. and pressure of greater than 650 mbar. The crude compound was purified by short path distillation (boiling point of 49° C. and p of 0.09 mbar). The title compound was obtained as a colorless liquid (7.84 g, 50%) which crystallized upon standing.

R_{f}=0.36 (heptane/EtOAc=7/3).

¹H NMR (CDCl₃, 300 MHz): δ=6.72 (br, s, 1H, N—H), 4.09 (dd, J₁=5.5 Hz, J₂=2.6 Hz, 2H, CH₂—NH), 3.92 (s, 2H, CH₂—OMe), 3.43 (s, 3H, OCH₃), 2.24 (t, J=2.6 Hz, 1H, alkyne CH). ¹³C-NMR (CDCl₃, 75 MHz): δ=169.14 (C═O), 79.11 (C-2′), 71.63 (C-2), 71.41 (C-3′), 59.04 (OCH₃), 28.26 (C-1′).

Gas chromatography was used to determine the t_{R} (min) of 6.42 under the conditions shown in the table below.

| | |
|---|---|
| Column | DB-5 (30 m × 0.32 mm, 0.25 μm film thickness) |
| Injector | Split, initial Temp. 250° C. |
| Split ratio | 60.243:1 |
| Split flow | 108.3 ml/min, gas type: hydrogen |
| Oven | 60° C., 1 min, 10° C./min, 290° C., 10 min |
| Inject-Temp | 250° C. |
| Detector (FID) | Detector Temp. 250° C. |
| Detector flow | H₂: 40.0 ml/min, air: 450 ml/min |
| Makeup flow | N₂: 45.0 ml/min |

Preparation of 2-methyl-2-butene (0.59 ml, 5.60 mmol, 2.8 equiv.) was added over 1 hour to a cold (0-5° C.) solution of BH₃*THF complex (1.0 M sol, 3.0 ml, 3.0 mmol, 1.5 equiv.) kept under N₂. The reaction mixture was stirred at this temperature for 30 minutes followed by the addition of 2-Methoxy-acetic acid propargylamide (255 mg, 2 mmol, 1.0 equiv.) dissolved in dry THF (1 ml) over 15 minutes. The ice-bath was removed and the reaction mixture was warmed to room temperature over 20 minutes. The reaction mixture was then heated at 35° C. for 1 hour. K₂CO₃ (0.55 g, 4 mmol, 2.0 equiv.) dissolved in degassed H₂O (1.2 ml) was added over 30 minutes to the reaction mixture. During the addition of the first half gas evolution was observed which seized during further addition. 6-Iodo-[3-methyl-4-(6-methyl-pyridine-3-yloxy)-phenylamino]-quinazoline (1.41 g, 3 mmol, 1.5 equiv.) was added in three portions giving a yellow suspension. PPh₃ (21 mg, 0.08 mmol, 4 mol %) and Pd(OAc)₂ (4.5 mg, 0.02 mmol, 1 mol %) were added each in one portion and the reaction mixture was heated to reflux (65-68° C.). After about 30 minutes a yellow solution was obtained and the reaction was monitored by HPLC assay. After 18 hours the reaction mixture was cooled to room temperature followed by the addition of half-saturated NaCl solution (10 ml) and EtOAc (10 ml). The organic phase was separated, washed with H₂O (5 ml) and concentrated at 50° C. and a pressure of less than 200 mbar. Purification by plug filtration, SiO₂, EtOAc/MeOH=9/1. The title compound was obtained as light yellow crystals (0.55 g, 59%). R_{f}=0.16 (EtOAc/MeOH=9/1). ¹H-NMR (CDCl₃, 250 MHz): δ=8.71 (s, 1H, H-2), 8.25 (d, J=1.7 Hz, 1H, H-8), 7.90(s, 1H, H-7), 7.82 (s, 1H, NH), 7.79 (s, 1H, H-5), 7.66 (d, J=2.5 Hz, 1H, H-4″), 7.54 (dd, J₁=8.7 Hz, J₂=2.6 Hz, 1H, H-5″), 7.15-7.07 (m, 2H, H-5′, H-6′), 6.91 (d, J=8.7 Hz, 1H, H-2′), 6.83 (bt, 1H, NH), 6.65 (d, J=15.9 Hz, 1H, H-9), 6.34 and 6.29 (dt, J₁=15.9 Hz, J₂=6.1 Hz, 1H, H-10), 4.14 (dt, J=6.1 Hz, 2H, CH₂OMe), 3.97 (s, 2H, CH₂NH), 3.45 (s, 3H, OCH₃), 2.53 (s, 3H, CH₃), 2.29 (s, 3H, CH₃). ¹³C-NMR (CDCl₃, 75 MHz): δ=169.76 (C═O), 157.90, 154.93, 152.367, 152.23, 150.90, 149.74, 139.34, 134.73, 134.63, 131.16, 130.77, 130.36, 128.85, 129.98, 125.47, 124.66, 123.65, 121.32, 119.51, 119.13, 115.39, 71.96, 59.26, 40.84, 23.57, 16.41.

Using reverse phase high performance liquid chromatography t_{R} (min) was found to be 6.02 for the title compound under the conditions shown in the following table.

| | |
|---|---|
| Symmetry Shield RP18 | 75 × 4.6 mm |
| Flow | 1.0 mL/min |
| Wavelength | 205/210/220/245 nm |
| Temp. | 25° C. |
| Injection Volume | 10 μL of a ca. 0.5% solution in ACN/H₂O 9/1 |
| Eluent B | ACN |
| Eluent C | 0.01 mmol NH₄OAc in H₂O pH = 6.0 |
| Gradient 0 min | B = 30%, C = 70% |
| Gradient 20 min | B = 85%, C = 15% |

Preparation of Crystal Form A

The E-2-Methoxy-N-(3-{4-[3-methyl-4-(6-methyl-pyridin-3-yloxy)-phenylamino]-quinazolin-6-yl}-allyl)-acetamide non-crystalline product was dissolved in boiling acetonitrile and then cooled to room temperature with stirring. The solids were then filtered and washed with cold acetonitrile to afford a crystalline powder.

### EXAMPLE 2

### Crystal Form B of E-2-Methoxy-N-(3-{4-[3-methyl-4-(6-methyl-pyridin-3-yloxy)-phenylamino]-quinazolin-6-yl}-allyl)-acetamide

The dimesylate salt of E-2-Methoxy-N-(3-{4-[3-methyl-4-(6-methyl-pyridin-3-yloxy)-phenylamino]-quinazolin-6-yl}-allyl)-acetamide is prepared as follows:

To 67.33 grams of the free base form E-2-Methoxy-N-(3-{4-[3-methyl-4-(6-methyl-pyridin-3-yloxy)-phenylamino]-quinazolin-6-yl}-allyl)-acetamide (prepared according to Example 1 above) in 400 mL of EtOH and 100 ml of CH₂Cl₂ at room temp was added dropwise a soln of 19.17 mL (2.05) equivs of methanesulfonic acid (CH₃SO₃H) in 100 ml of acetonitrile. The mixture was slurried at room temperature for 15 minutes then the methylene chloride (˜100 ml) was removed. An additional 600 mL of acetonitrile was added to complete crystallization and the mixture slurried for 2 hours. The crystals were filtered under a nitrogen atmosphere and washed with 100 ml of acetonitrile. The dimesylate salt (94.48 grams) was produced in 99% yield.

The dimesylate salt produced according to the method of the preceding paragraph (90 g) was dissolved in water (˜550 mL). Chloroform was added (˜500 mL) to the solution followed by 1N NaOH until a white suspension/precipitate was observed (pH ˜13-14). The addition of chloroform before NaOH reduced gumming as the precipitate formed. The mixture was transferred to a separatory funnel (2 L) and the free base was extracted with three portions of chloroform (˜300 mL). The extracts were combined (˜1.3 L), washed with water (˜500 mL), dried with anhydrous magnesium sulfate, and then filtered. The chloroform filtrate was concentrated in vacuo to provide a yellow amorphous solid/oil. This material was reslurried in ethyl acetate overnight resulting in a white solid. This material was then filtered, washed with cold ethyl acetate, and then dried in a vacuum oven at 45° C. to yield a white crystalline solid (˜59 g). The free base was characterized by polarizing light microscopy (PLM), powder X-ray diffraction (PXRD), and differential scanning calorimetry (DSC). It is in the form of a needle, and displays three endothermic events by DSC (DSC melting points: 125° C., 160° C., and 167° C.)

### EXAMPLE 3

### Crystal Form C of E-2-Methoxy-N-(3-{4-[3-Methyl-4-(6-methyl-pyrodin-3-yloxy)-phenylamino]-quinazolin-6-yl}-allyl)-acetamide

Form C was prepared by combining Form B and methylene chloride, and then concentrating to yield a foam. The foam was then slurried in acetonitrile for approximately three hours at room temperature and filtered to afford Form C.

### EXAMPLE 4

### Crystal Form F of E-2-Methoxy-N-(3-{4-[3-Methyl-4-(6-Methyl-Pyridin-3-Yloxy)-phenylamino]-quinazolin-6-yl}-allyl)-acetamide

Form F was prepared by combining Form B, as prepared in Example 2, and ethyl acetate to form a slurry. To this slurry, hydrogen peroxide (30%) and water were added, and the resulting mixture was stirred overnight. The solids were isolated by filtration, rinsed with water and acetone, and vacuum-dried at 40-45° C. to afford Form F.

### EXAMPLE 5

### Crystal Form G of E-2-Methoxy-N-(3-{4-[3-methyl-4-(6-methyl-pyridin-3-yloxy)-phenylamino]-quinazolin-6-yl}-allyl)-acetamide

Form G was prepared by forming a solution of free base (20 grams), formed in accordance with Example 1, methanol (10 mL) and methyl-tetrahydrofuran (90 mL) at 60° C. The solution was allowed to cool to room temperature, and after reaching 23° C. formed a slurry. The solids were isolated by filtration and washed using methyl-tetrahydrofuran to yield Form G.

### EXAMPLE 6

### Crystal Form H of E-2-Methoxy-N-(3-{4-[3-methyl-4-(6-methyl-pyridin-3-yloxy)-phenylamino]-quinazolin-6-yl}-allyl)-acetamide

To a solution of E-2-Methoxy-N-(3-{4-[3-methyl-4-(6-methyl-pyridin-3-yloxy)-phenylamino]-quinazolin-6-yl}-allyl)-acetamide in hot THF/acetone (5/100) two equivalents of succinic acid were added. Crystals slowly formed as the solution cooled. After slurrying overnight, the crystals were filtered and rinsed with acetone. The product was isolated as a white solid and verified as the sesquisuccinate complex of E-2-Methoxy-N-(3-{4-[3-methyl-4-(6-methyl-pyridin-3-yloxy)-phenylamino]-quinazolin-6-yl}-allyl)-acetamide by CHN analysis. Calculated: C=61.29, H=5.61, N=10.83, Experimental: C=61.04, H=5.61, N=10.85.

The thus formed sesquisuccinate complex (5 grams) was mixed with water (25 mL) and chloroform (25 mL). To this mixture 1.1 equivalents of sodium hydroxide (1 normal aqueous) was added and stirred until two layers had formed. The mixture was transferred into a separatory funnel and the layers were separated. The contents of the funnel were mixed well and the layers separated. The extraction was repeated a second time with an additional aliquot of chloroform (25 mL). The water layer was then discarded and the combined chloroform layers (˜75 mL) were placed back into the separatory funnel with some water (25 mL). The contents of the funnel were mixed well and the layers separated. The water layer was discarded and the chloroform layer was placed into a single-neck round bottom flask.

The chloroform was removed using rotary evaporation to yield a yellow oil. Ethyl acetate was added (125 mL) and stirred with the oil to yield a thick slurry. After stirring for approximately one day, the slurry was isolated via vacuum filtration using a Buchner funnel fitted with a paper filter (Whatman #2). The solids were rinsed with ethyl acetate (25 mL), and then placed into a crystallizing dish. The dish and solids were placed into a vacuum oven at 45° C. to dry overnight to afford a pale yellow powder.

1. A crystal form of E-2-Methoxy-N-(3-{4-[3-methyl-4-(6-methyl-pyridin-3-yloxy)-phenylamino]-quinazolin-6-yl}-allyl)-acetamide.

2. A crystal form of E-2-Methoxy-N-(3-{4-[3-methyl-4-(6-methyl-pyridin-3-yloxy)-phenylamino]-quinazolin-6-yl}-allyl)-acetamide selected from the group consisting of Form A, Form C, Form F, Form G and Form H.

3. A crystal form in accordance with claim 2 wherein said form is Form A, having an X-ray powder diffraction spectrum having characteristic peaks, expressed in degrees (2-theta), as follows:

| 2-Theta (Degree) | Rel. % Intensity |
|---|---|
| 6.3 | 100.0 |
| 16.1 | 51.7 |
| 19.0 | 52.6 |
| 23.2 | 64.4 |
| 24.7 | 99.6 |

4. A crystal form in accordance with claim 2 wherein said form is selected from the group consisting of:
(a) Form A, having an X-ray powder diffraction spectrum having characteristic peaks, expressed in degrees (2-theta), as follows:

| 2-Theta (Degree) | Rel. % Intensity |
|---|---|
| 6.3 | 100.0 |
| 9.7 | 44.2 |
| 13.5 | 44.2 |
| 16.1 | 51.7 |
| 19.0 | 52.6 |
| 21.2 | 41.3 |
| 21.4 | 51.6 |
| 23.2 | 64.4 |
| 24.7 | 99.6 |
| 26.5 | 43.7 |

(b) form a having an x-ray powder diffraction spectrum having characteristic peaks, expressed in degrees (2-theta), as follows:

| 2-Theta (degree) | Rel. % intensity |
|---|---|
| 6.3 | 100.0 |
| 9.7 | 44.2 |
| 10.1 | 30.9 |
| 11.5 | 26.6 |
| 12.6 | 41.1 |
| 13.5 | 44.2 |
| 15.0 | 19.5 |
| 16.1 | 51.7 |
| 18.1 | 41.0 |
| 19.0 | 52.6 |
| 19.5 | 12.6 |
| 20.1 | 25.9 |
| 21.2 | 41.3 |
| 21.4 | 51.6 |
| 21.7 | 31.0 |
| 22.2 | 15.4 |
| 22.8 | 33.4 |
| 23.2 | 64.4 |
| 23.7 | 22.9 |
| 24.3 | 27.9 |
| 24.7 | 99.6 |
| 25.6 | 18.0 |
| 26.5 | 43.7 |
| 27.1 | 26.9 |
| 27.5 | 17.2 |
| 28.4 | 21.7 |
| 29.5 | 23.0 |
| 30.7 | 16.1 |

(c) Form C having an X-ray powder diffraction spectrum having characteristic peaks, expressed in degrees (2-theta), as follows:

| 2-Theta (Degree) | Rel. % Intensity |
|---|---|
| 4.8 | 100.0 |
| 9.7 | 33.2 |
| 14.6 | 35.9 |
| 18.4 | 29.1 |
| 25.9 | 33.2 |

(d) Form C having an X-ray powder diffraction spectrum having characteristic peaks, expressed in degrees (2-theta), as follows:

| 2-Theta (Degree) | Rel. % Intensity |
|---|---|
| 4.8 | 100.0 |
| 9.7 | 3.2 |
| 14.1 | 23.1 |
| 14.6 | 35.9 |
| 18.4 | 29.1 |
| 18.7 | 24.0 |
| 22.3 | 25.7 |
| 22.9 | 20.1 |
| 24.0 | 19.0 |
| 25.9 | 33.2 |

(e) Form C having an X-ray powder diffraction spectrum having characteristic peaks, expressed in degrees (2-theta), as follows:

| 2-Theta (degree) | Rel. % intensity |
|---|---|
| 4.8 | 100.0 |
| 8.3 | 8.9 |
| 9.7 | 33.2 |
| 10.5 | 12.5 |
| 14.1 | 23.1 |
| 14.6 | 35.9 |
| 16.1 | 7.9 |
| 16.9 | 4.9 |
| 18.4 | 29.1 |
| 18.7 | 24.0 |
| 19.4 | 7.1 |
| 19.7 | 6.3 |
| 21.2 | 12.5 |
| 22.0 | 15.1 |
| 22.3 | 25.7 |
| 22.9 | 20.1 |
| 23.7 | 14.1 |
| 24.0 | 19.0 |
| 24.6 | 6.3 |
| 25.9 | 33.2 |
| 26.7 | 11.0 |
| 27.0 | 10.4 |
| 28.6 | 6.5 |
| 29.0 | 6.7 |
| 30.2 | 9.3 |
| 30.7 | 5.5 |
| 32.1 | 5.7 |
| 33.8 | 4.2 |

(f) Form F having an X-ray powder diffraction spectrum having characteristic peaks, expressed in degrees (2-theta), as follows:

| 2-Theta (Degree) | Rel. % Intensity |
|---|---|
| 10.9 | 61.0 |
| 13.1 | 92.1 |
| 15.3 | 100.0 |
| 19.3 | 88.1 |
| 21.1 | 50.8 |

(g) Form F having an X-ray powder diffraction spectrum having characteristic peaks, expressed in degrees (2-theta), as follows:

| 2-Theta (Degree) | Rel. % Intensity |
|---|---|
| 10.2 | 38.0 |
| 10.9 | 61.0 |
| 13.1 | 93.1 |
| 15.3 | 100.0 |
| 16.5 | 45.5 |
| 18.5 | 42.2 |
| 19.0 | 44.0 |
| 19.3 | 88.1 |
| 21.1 | 50.8 |
| 23.3 | 39.8 |

(h) Form F having an X-ray powder diffraction spectrum having characteristic peaks, expressed in degrees (2-theta), as follows:

| 2-Theta (degree) | Rel. % intensity |
|---|---|
| 5.4 | 27.4 |
| 6.1 | 30.4 |
| 9.6 | 27.9 |
| 10.2 | 38.0 |
| 10.9 | 61.0 |
| 13.1 | 92.1 |
| 15.3 | 100.0 |
| 16.5 | 45.5 |
| 17.9 | 27.6 |
| 18.5 | 42.2 |
| 19.0 | 44.0 |
| 19.3 | 88.1 |
| 20.6 | 31.7 |
| 21.1 | 50.8 |
| 21.7 | 20.2 |
| 22.5 | 32.2 |
| 23.3 | 39.8 |
| 23.8 | 36.8 |
| 24.4 | 29.0 |
| 24.7 | 43.8 |
| 25.5 | 28.0 |
| 26.1 | 25.7 |
| 26.6 | 25.7 |
| 31.3 | 20.2 |
| 32.5 | 17.4 |

(i) Form G having an X-ray powder diffraction spectrum having characteristic peaks, expressed in degrees (2-theta), as follows:

| 2-Theta (Degree) | Rel. % Intensity |
|---|---|
| 7.8 | 100.0 |
| 15.0 | 75.9 |
| 16.7 | 40.0 |
| 19.3 | 41.2 |
| 24.7 | 35.2 |

(j) Form G having an X-ray powder diffraction spectrum having characteristic peaks, expressed in degrees (2-theta), as follows:

| 2-Theta (Degree) | Rel. % Intensity |
|---|---|
| 7.8 | 100.0 |
| 14.5 | 16.8 |
| 15.0 | 75.9 |
| 16.7 | 40.0 |
| 19.3 | 41.2 |
| 21.8 | 15.5 |
| 22.7 | 22.7 |
| 23.2 | 19.9 |
| 23.3 | 22.5 |
| 24.7 | 35.2 |

(k) Form G having an X-ray powder diffraction spectrum having characteristic peaks, expressed in degrees (2 theta), as follows:

| 2-Theta (degree) | Rel. % intensity |
|---|---|
| 7.8 | 100.0 |
| 10.4 | 6.9 |
| 12.2 | 6.4 |
| 14.5 | 16.8 |
| 15.0 | 75.9 |
| 15.8 | 11.7 |
| 16.7 | 40.0 |
| 17.4 | 7.8 |
| 18.2 | 6.8 |
| 19.3 | 41.2 |
| 21.0 | 11.1 |
| 21.8 | 15.5 |
| 22.2 | 10.5 |
| 22.7 | 22.7 |
| 23.2 | 19.9 |
| 23.3 | 22.5 |
| 23.8 | 12.7 |
| 24.7 | 35.2 |
| 25.7 | 13.0 |
| 26.7 | 6.4 |
| 27.3 | 14.9 |
| 27.6 | 12.1 |
| 28.2 | 6.0 |
| 28.9 | 10.8 |
| 30.0 | 8.8 |
| 30.9 | 9.2 |
| 31.7 | 7.2 |
| 37.8 | 8.7 |

(l) Form H having an X-ray powder diffraction spectrum having characteristic peaks, expressed in degrees (2-theta), as follows:

| 2-Theta (Degree) | Rel. % Intensity |
|---|---|
| 4.6 | 100.0 |
| 15.1 | 42.9 |
| 17.7 | 19.1 |
| 21.4 | 15.7 |
| 22.7 | 19.8 |

(m) Form H having an X-ray powder diffraction spectrum having characteristic peaks, expressed in degrees (2-theta), as follows:

| 2-Theta (Degree) | Rel. % Intensity |
|---|---|
| 4.6 | 100.0 |
| 14.0 | 15.1 |
| 15.1 | 42.9 |
| 17.7 | 19.1 |
| 19.4 | 15.0 |
| 20.4 | 10.1 |
| 21.0 | 10.5 |
| 21.4 | 15.7 |
| 22.7 | 19.8 |
| 23.9 | 15.4 |

and
(n) Form H having an X-ray powder diffraction spectrum having characteristic peaks, expressed in degrees (2 theta), as follows:

| 2-Theta (degree) | Rel. % intensity |
|---|---|
| 4.6 | 100.0 |
| 7.1 | 5.1 |
| 8.1 | 6.2 |
| 9.3 | 5.2 |
| 11.5 | 6.3 |
| 14.0 | 15.1 |
| 15.1 | 42.9 |
| 15.8 | 5.2 |
| 16.4 | 5.3 |
| 17.7 | 19.1 |
| 19.4 | 15.0 |
| 20.4 | 10.1 |
| 21.0 | 10.5 |
| 21.4 | 15.7 |
| 21.9 | 8.8 |
| 22.7 | 19.8 |
| 23.9 | 15.4 |
| 25.0 | 5.6 |
| 25.8 | 5.2 |
| 26.9 | 6.4 |
| 27.7 | 7.5 |
| 29.7 | 5.2 |
| 30.6 | 3.7 |
| 31.2 | 4.1 |
| 33.6 | 3.5 |

5. A crystal form in accordance with claim 2 wherein said form is Form A having a formula weight of 469.54; a crystal size of 0.24 mm×0.08 mm×0.06 mm, a space group P21 (1)/n monoclinic and unit cell dimensions: a=9.456 Å, b=9.237 Å, c=27.947 Å, α=90°, β=92.97° and □=90°.

6. A crystal form in accordance with claim 2 wherein said Form is Form A, characterized by solid-state 13C nuclear magnetic resonance having the following chemical shifts expressed in parts per million: 172.1, 158.6, 153.7, 149.6, 147.7, 138.8, 135.0, 132.4, 129.9, 125.8, 123.6, 120.9, 119.4, 118.4, 117.1, 71.3, 58.1, 44.0, 20.7 and 16.6.

7. A composition comprising the crystal form of claim 1.

8. A pharmaceutical composition comprising the crystal form of claim 1.
